(19) United States
(12) Patent Application Publication
(10) Pub. No.: US 2015/0314023 A1
(43) Pub. Date: Nov. 5, 2015

(54) TOOTHBRUSH AND RAZOR HOLDER AND SYSTEM FOR PROLONGING THE USEFUL LIFE OF TOOTHBRUSHES AND RAZORS

(71) Applicant: WILLIAM R. NEW, FAYETTEVILLE, GA (US)

(72) Inventor: WILLIAM R. NEW, FAYETTEVILLE, GA (US)

(21) Appl. No.: 14/796,975

(22) Filed: Jul. 10, 2015

Related U.S. Application Data
(63) Continuation of application No. 29/449,958, filed on Mar. 15, 2013, now Pat. No. D734054.

(51) Int. Cl. A61L 2/08; A61L 2/18

(52) U.S. Cl. CPC A61L 2/084; A61L 2/18

(57) ABSTRACT

A system for and method of sanitizing a toothbrush and a razor is provided. The system includes a base member that is configured to support a pair of containers such that at least some light emitted from a sanitizing light propagates into an interior area of each container so as to sanitize the bristles of the toothbrush and the blades of the razor. The method includes pouring a first cleaning fluid, such as mouthwash, into the first container and a second cleaning fluid, such as rubbing alcohol, into the second container. The toothbrush and razor are then placed head-first into the first and second containers, respectively, such that the bristles of the toothbrush and the blades of the razor are completely submerged in respective cleaning fluids. A sanitizing light is then directed towards the bristles of the toothbrush and the blades of the razor.

### CROSS-REFERENCE TO RELATED APPLICATIONS

This application claims priority pursuant to 35 U.S.C. 119(e) to co-pending U.S. Design patent application Ser. No. 29/449,958, filed Mar. 15, 2013, the entire disclosure of which is incorporated herein by reference.

### FIELD OF THE INVENTION

The present invention relates generally to sanitizing systems. More specifically, the present invention is concerned with system and method for sanitizing and/or otherwise preserving the life of objects such as toothbrushes and razors.

### BACKGROUND OF THE INVENTION

Millions of people unknowingly use dirty and/or broken-down toothbrushes and razors on a daily basis. For instance, people rarely wash a toothbrush or a razor sufficiently after use to remove all of the plaque or other debris that becomes embedded in the bristles of a toothbrush or the hair and skin particles that become embedded between the blades of a razor. Furthermore, whether a toothbrush or razor is placed in a drawer or a cabinet or is simply left out next to a sink, particles in the air and/or on surfaces can contact and cling to the toothbrush and/or razor. These particles can contain harmful chemicals or microorganisms such as bacteria. Subsequent use of the toothbrush and/or razor can then introduce these harmful chemicals and/or microorganisms into a user's mouth and/or into the user's blood stream through an inadvertent cut caused when shaving. Furthermore, contact with air can cause blades to rust prematurely, especially if the blades are not properly dried after use.

Consequently, it would be beneficial to have a system that is capable of sanitizing a toothbrush and/or a razor between uses. It would further be beneficial if the system prevented air and/or other objects, such as surfaces that might contain harmful chemicals and/or microorganisms, from contacting the bristles of the toothbrush and/or the blades of the razor.

### SUMMARY OF THE INVENTION

The present invention comprises a base member having sanitizing light and a recessed area. The recessed area is configured to receive part of a first and a second container so as to hold the containers in an upright configuration while the sanitizing light is being directed towards them. Each container is at least partially made of transparent, semi-transparent, and/or otherwise translucent material such that light from the sanitizing light is able to propagate into an interior area of each container.

In some embodiments, one of the containers is taller than the other container. The taller container is particularly suited for holding a toothbrush, while the shorter container is particularly suited for holding a razor. The toothbrush container is partially filled with mouthwash (or some other suitable sanitizing or cleaning fluid) to a level sufficient to submerge the head of a toothbrush placed head-down in the container. The razor container is partially filled with rubbing alcohol (or some other suitable sanitizing or cleaning fluid) to a level sufficient to submerge the head of a razor placed head-down in the container. Lids of the containers minimize evaporation of the fluids, while the toothbrush/razor is submerged. The toothbrush and/or razor are stored in the containers between uses to sanitize and help prolong the useful life of the same.

In a preferred embodiment, a sanitizing light source is housed in the base member and projected up through a lens portion of the base member directly below the containers. A push button on the base member is used to turn on and off the light source. In some preferred embodiments the light source includes circuitry to automatically turn off the light source after a predetermined period. In some such embodiments, the predetermined period is based upon an amount of time deemed necessary to satisfactorily sanitize the toothbrush and/or razor with the light source.

The present invention further comprises a method of sanitizing and/or otherwise prolonging the life of an object such as a toothbrush and/or a razor. The method includes pouring a fluid, such as mouthwash or rubbing alcohol, into a container and placing a toothbrush or a razor, head first, into the container so that the head of the toothbrush or razor is completely submerged by the fluid. The method further includes placing the container relative to a light source such that when the light source is turned on, light emitting from the light source propagates into the container so as to sanitize the blades of the razor and/or the bristles of the toothbrush.

In some embodiments, the light source is powered by replaceable batteries. In other embodiments, the light source is powered by an electrical cord connected to a separate power source such as an a/c wall outlet.

The foregoing and other objects are intended to be illustrative of the invention and are not meant in a limiting sense. Many possible embodiments of the invention may be made and will be readily evident upon a study of the following specification and accompanying drawings comprising a part thereof. Various features and subcombinations of invention may be employed without reference to other features and subcombinations. Other objects and advantages of this invention will become apparent from the following description taken in connection with the accompanying drawings, wherein is set forth by way of illustration and example, an embodiment of this invention and various features thereof.

### BRIEF DESCRIPTION OF THE DRAWINGS

A preferred embodiment of the invention, illustrative of the best mode in which the applicant has contemplated applying the principles, is set forth in the following description and is shown in the drawings and is particularly and distinctly pointed out and set forth in the appended claims.

FIG. 1 is a perspective view of a preferred embodiment of the present invention.

FIG. 2 is a top plan view of the embodiment of FIG. 1.

FIG. 3 is a front elevation view of the embodiment of FIG. 1.

FIG. 4 is a bottom view of the embodiment of FIG. 1.

FIG. 5 is a right side elevation view of the embodiment of FIG. 1.

FIG. 6 is a left side elevation view of the embodiment of FIG. 1.

FIG. 7 is a rear side elevation view of the embodiment of FIG. 1.

FIG. 8 is another perspective view of the embodiment of FIG. 1.

FIG. 9 is an exploded perspective view of the embodiment of FIG. 1.

FIG. 10 is a perspective view of an alternative embodiment of the present invention.

FIG. 11 is a perspective view of yet another alternative embodiment of the present invention.

FIG. 12 is a perspective cut-away view showing the containers removed from the base member.

FIG. 13 is a partial elevation cut-away view showing the containers being received by the base member and the light source emitting light towards the containers.

FIG. 14 is an isolated view on an enlarged scale of a portion of FIG. 13.

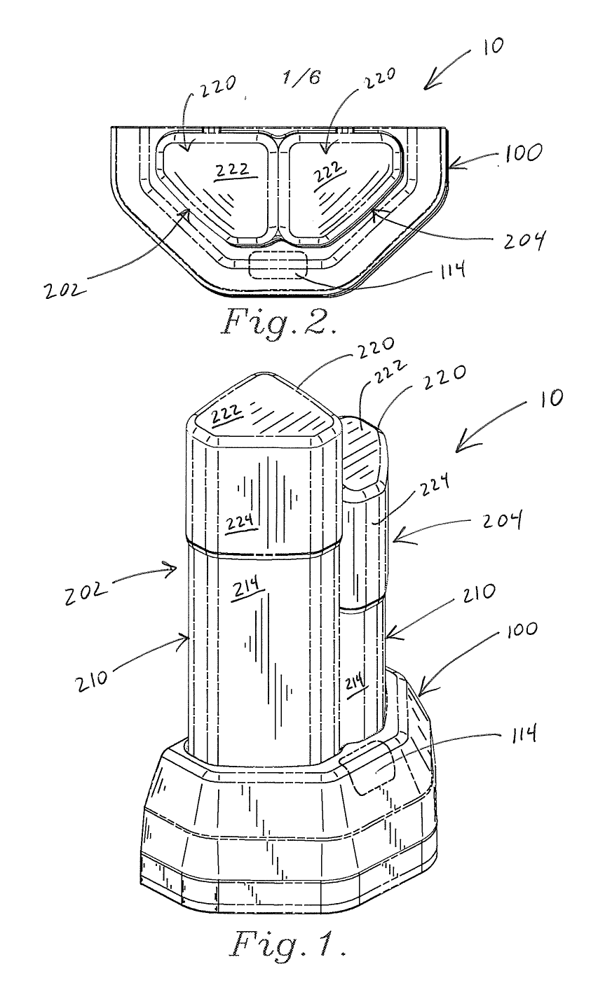

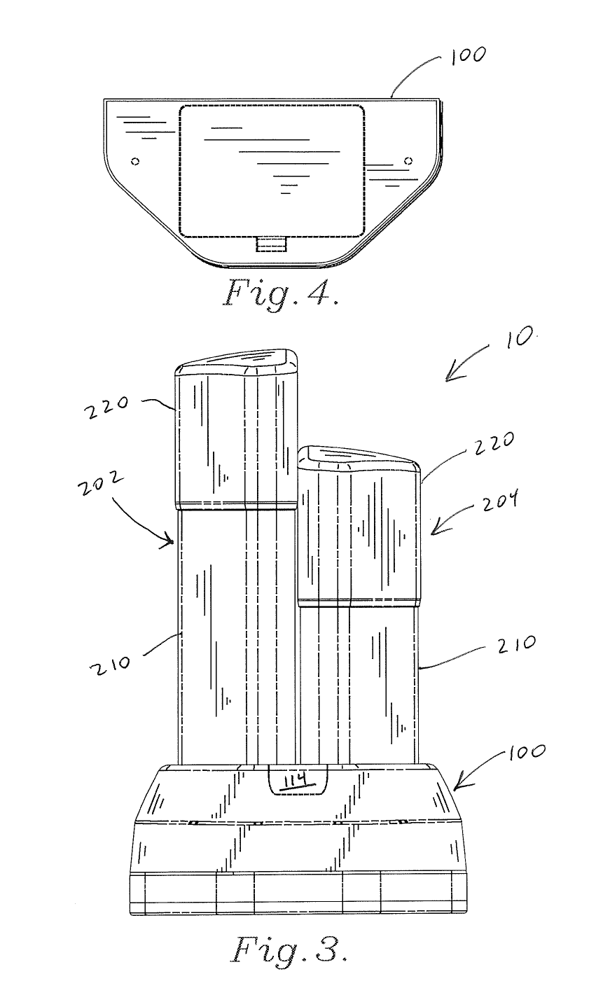

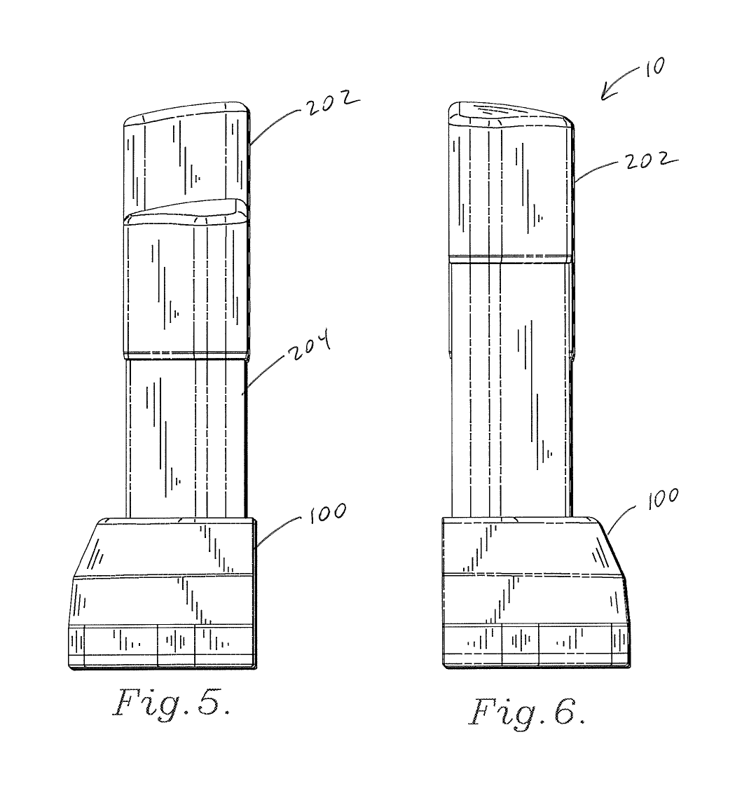

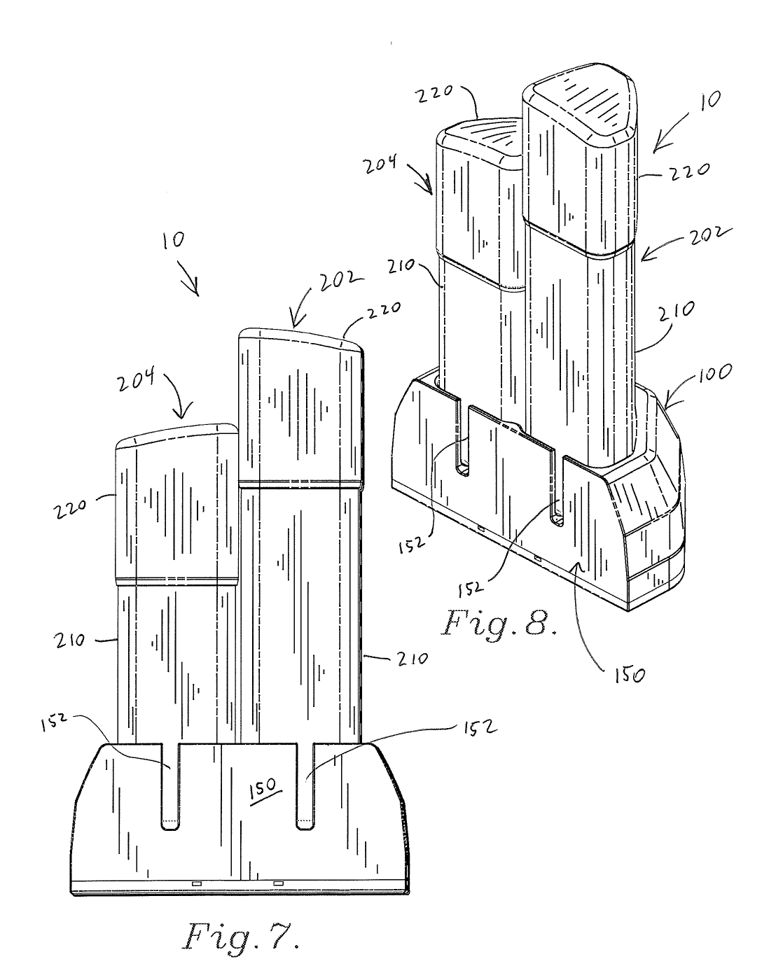

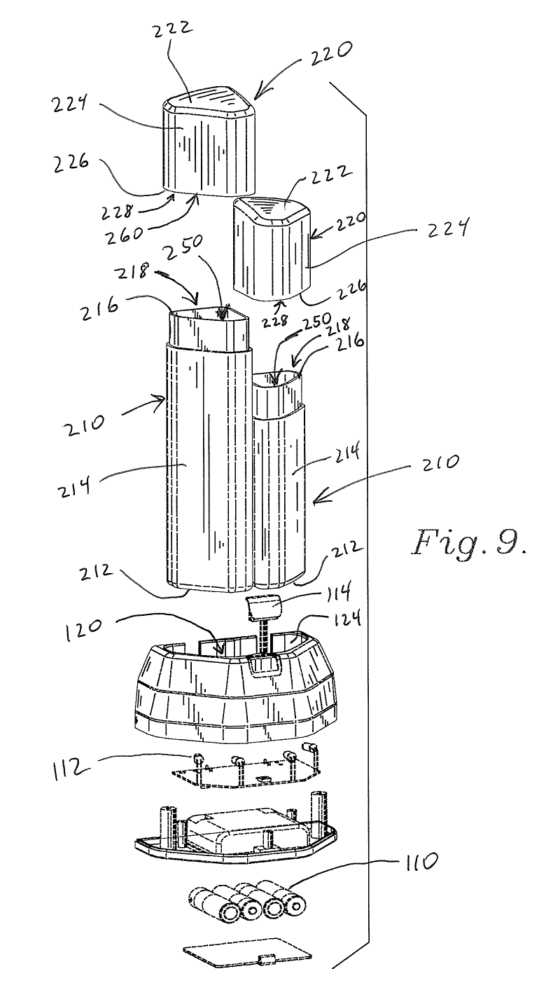

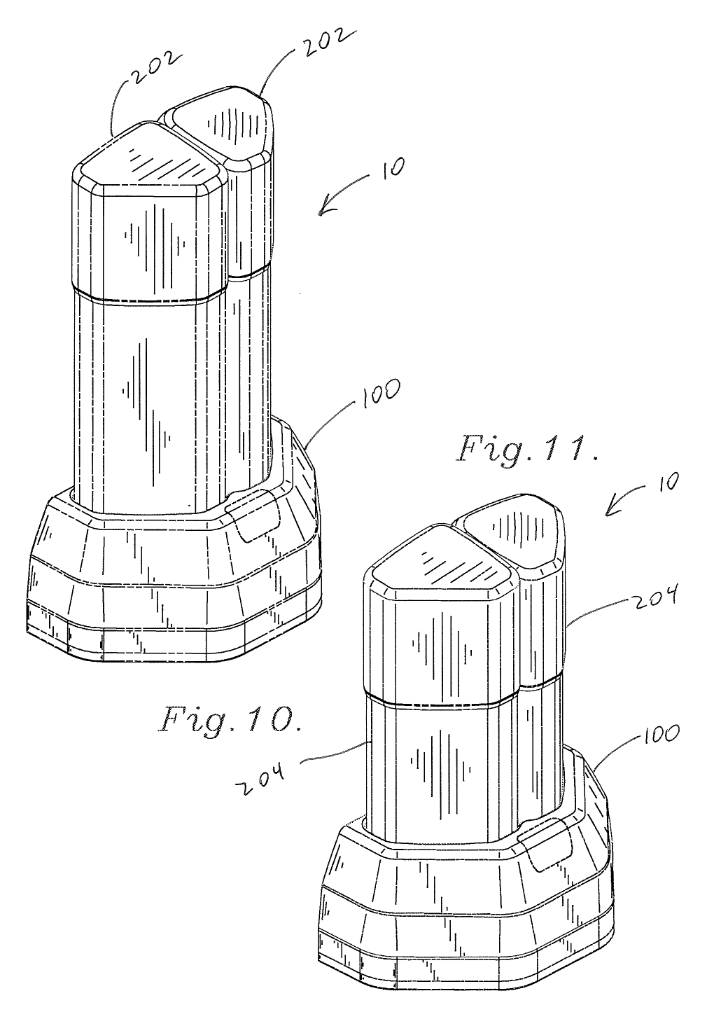

### DETAILED DESCRIPTION OF A PREFERRED EMBODIMENT

As required, a detailed embodiment of the present invention is disclosed herein; however, it is to be understood that the disclosed embodiment is merely exemplary of the principles of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Referring to FIG. 1, a preferred embodiment of the system of the present invention includes a base member 100, a first container 202, and a second container 204. Each container 202, 204 includes a lid portion 220 and a bottom portion 210.

The bottom portion 210 of each container 202, 204 includes a bottom panel 212, a top lip 216 vertically displaced from the bottom panel 212, and a continuous sidewall 214 extending between the bottom panel 212 and the top lip 216 so as to define a first interior area 250 that is capable of holding a variety of fluids such as water, mouthwash, rubbing alcohol, or the like. The top lip 216 of the bottom portion 210 defines a top opening 218 that is in communication with the interior area 250 such that liquid can be poured into or out of the first interior areas 250 of the first 202 and second 204 containers through respective top openings 218 of the first 202 and second 204 containers.

The lid portion 220 of each container 202, 204 includes a bottom lip 226 that is configured to selectively couple to the top lip 116 of the bottom portion 210 of its respective container 202, 204 so as to selectively couple respective top 220 and bottom 210 portions together. In some embodiments, selectively coupling top 220 and bottom 210 portions together creates a seal that prevents or inhibits liquids from being poured into or out of the respective container 202, 204. In some such embodiments, the seal also prevents or inhibits other fluids, such as air, from flowing into or out of the respective container 202, 204. In this way, selectively coupling the lid portion 220 of a container 202, 204 to the bottom portion of the respective container 202, 204 minimizes evaporation of liquid within the container 202, 204.

Some embodiments of lid portions 220 further include a top panel 222 vertically displaced from the bottom lip 226 of the lid portion 220. In some such embodiments, a continuous sidewall 224 extends between the top panel 222 and the bottom lip 226 so as to define a second interior area 260.

In some embodiments of the present invention, the first container 202 is configured to hold a toothbrush entirely within the first interior area 250. In other embodiments, a first portion of the toothbrush is positioned within the first interior area 250 and a second portion of the toothbrush extends out of the first interior area 250 through the top opening 218. In this way, a user is able to more easily remove the toothbrush from the first container 202 without requiring the user to reach into the first interior area 250 of the first container 202. In some such embodiments, the bottom opening 228 and the second interior area 260 of the lid portion 220 of the first container 202 are configured such that the second portion of the toothbrush extends through the bottom opening 228 into the second interior area 260 when the first portion of the toothbrush is positioned in the bottom portion 210 of the first container 202 and the lid portion 220 is selectively coupled to the bottom portion 210. In some such embodiments, the first portion of the toothbrush includes the bristles of the toothbrush and the first interior area 250 of the first container 202 is configured so as to be able to retain a sufficient amount of liquid such that a user can completely submerge the bristles of the toothbrush within a liquid, such as mouthwash, so as to sanitize and/or otherwise prolong the life of the toothbrush. In other such embodiments, the second portion of the toothbrush is a portion of the handle of the toothbrush.

In other embodiments of the present invention, the second container 204 is configured to hold a razor entirely within the first interior area 250. In other embodiments, a first portion of the razor is positioned within the first interior area 250 and a second portion of the razor extends out of the first interior area 250 through the top opening 218. In this way, a user is able to more easily remove the razor from the second container 204 without requiring the user to reach into the first interior area 250 of the second container 204. In some such embodiments, the bottom opening 228 and the second interior area 260 of the lid portion 220 of the second container 204 are configured such that the second portion of the razor extends through the bottom opening 228 into the second interior area 260 when the first portion of the razor is positioned in the bottom portion 210 of the second container 204 and the lid portion 220 is selectively coupled to the bottom portion 210. In some such embodiments, the first portion of the razor includes the blades of the razor and the first interior area 250 of the second container 204 is configured so as to be able to retain a sufficient amount of liquid such that a user can completely submerge the blades of the razor within a liquid, such as rubbing alcohol, so as to sanitize and/or otherwise prolong the life of the razor. In other such embodiments, the second portion of the razor is a portion of the handle of the razor.

It will be appreciated that, in some embodiments, the first container 202 is taller than the second container 204 so that the first container is configured to hold a toothbrush and the second container is configured to hold a razor. It will further be appreciated that, in other embodiments, the first container 202 and the second container 204 are the same height. For instance, in some such embodiments, the second container 204 is the same height as the first container 202 so that each container is configured to hold a toothbrush. In other such embodiments, the first container 202 is the same height as the second container 204 so that each container is configured to hold a razor. It will still further be appreciated that, in still other embodiments, the present invention includes a first container 202 but not a second container 204. It will yet further be appreciated that, in yet other embodiments, the present invention includes a plurality of containers in excess of two containers.

Some embodiments of the base member 100 include a recessed area 120 having a bottom surface 122 and sidewalls 124 extending vertically from the bottom surface 122. In some such embodiments, the recessed area 120 of the base member 100 is configured to receive at least a portion of the first container 202 such that the first container 202 is held in an upright configuration when the first container 202 is received by the recessed area 120 of the base member 100. In other such embodiments, the recessed area 120 is configured to receive at least a portion of the first container 202 and the second container 204 such that the first 202 and second 204 containers are each held in an upright configuration when they are received by the recessed area 120. In still other such embodiments, the recessed area 120 is configured such that first 202 and the second 204 containers are positioned adjacent to each other when they are both received by the recessed area 120.

It will be appreciated that, in some embodiments, the base member 100 includes one or more recessed area that is configured to receive one or more container such that the base member 100 is capable of supporting a plurality of containers in an upright configuration. It will further be appreciated that, in other embodiments, each container 202, 204 is capable of being removed from the base member 110 such that the container 202, 204 can be overturned so as to enable ease of emptying the container and/or cleaning the container.

Some embodiments of the present invention include at least one light source 112. In some embodiment, the light source is a sanitizing light source that is capable of emitting light with sufficient power to kill germs or otherwise sanitize an object and/or a liquid. The light source is directed towards the first 202 and second 204 containers. At least a portion of each container 202, 204 is made of transparent, semi-transparent, or otherwise fully or partially translucent material so that at least some of the light that is directed towards the respective container 202, 204 is capable of propagating into the interior area 250 of the container 202, 204. In this way, the light is able to sanitize liquid and/or an object that is positioned within the first interior area 250 of the container 202, 204.

In some embodiments, the light source 112 is positioned within the base member 100. In other embodiments, a lens 130 is positioned between the light source 112 and the first 202 and second 204 containers such that light emitted from the light source 112 propagates through the lens 130 prior to reaching the first 202 and second 204 containers. In some such embodiments, the light source 112 and the lens 130 are each part of the base member 100.

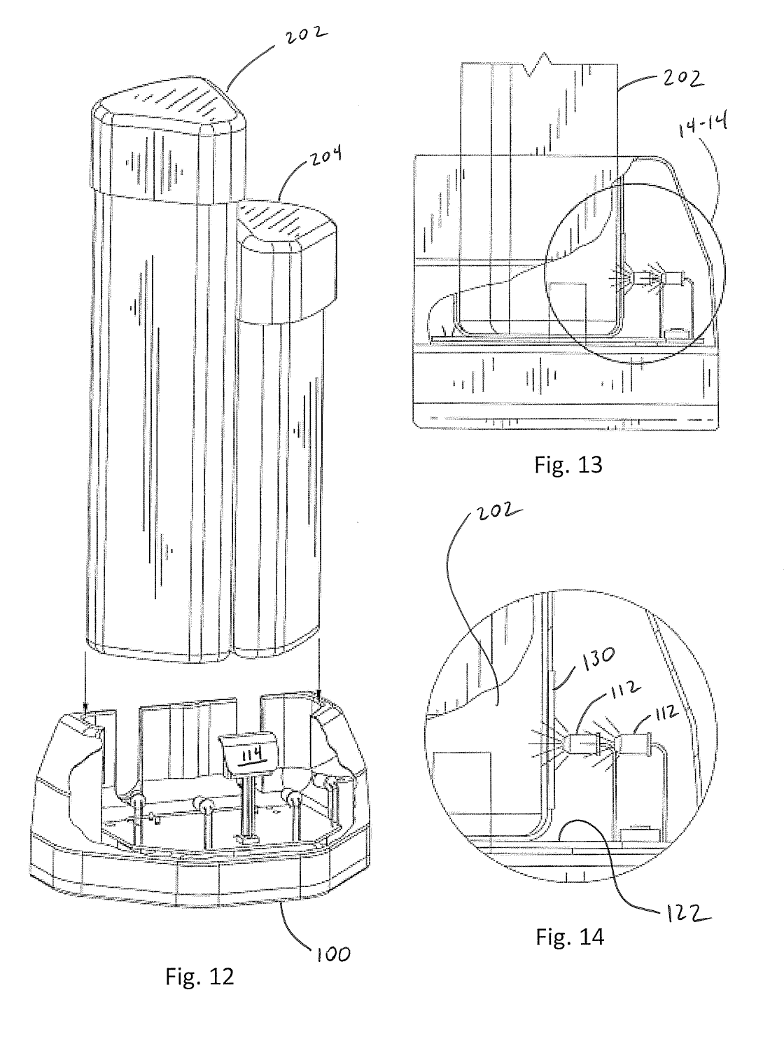

In a preferred embodiment, such as shown in FIGS. 12-14, a light source 112 is positioned proximately adjacent to bottom portions 210 of a first 202 and a second 204 container such that at least some of the light being emitted from the light source 112 propagates through the continuous sidewall 214 of each container 202, 204. In some such embodiments, the light source is slightly vertically displaced from the bottom panel 212 of each container 202, 204 such that the light is more likely to come in contact with portions of objects that are placed near the bottom of the respective interior areas 250 of each container 202, 204, such as bristles of a toothbrush and/or blades of a razor. In some such embodiments, the light source 112 is a single light source, such as a single light emitting diode or other discrete device for emitting light. In other such embodiments, the light source 112 includes a plurality of light sources, such as a plurality of light emitting diodes or the like.

In another preferred embodiment, not shown, the light source 112 is positioned directly below the containers 202, 204 and at least some of the light being emitted from the light source 112 is directed upward towards the containers 202, 204 such that at least some of the light from the light source 112 propagates through the bottom panel of each container 202, 204. In this way, the light is more likely to come in contact with portions of objects that are placed near the bottom of the respective interior areas 250 of each container 202, 204. In some such embodiments, the light source 112 is a single light source, such as a single light emitting diode or other discrete device for emitting light. In other such embodiments, the light source 112 includes a plurality of light sources, such as a plurality of light emitting diodes or the like.

In some embodiments, the base member 100 further includes a power source 110, such as one or more batteries and/or an electrical cord that is capable of connecting to an external power source. In other embodiments, the base member 100 further includes at least one button 114, such as a push button 114. In some such embodiments, the button 114 is in electrical communication with the power source 110 and the light source 112 so as to enable the button 114 to turn the light source 112 on and off by connecting and disconnecting the light source 112 from the power source 110. In other embodiments, the base member 200 further includes circuitry in electrical communication with the power source 110 and the light source 112. In some such embodiments, the circuitry is configured to automatically disconnect the light source 112 from the power source 110 after a predetermined period of time so as to shut the light source 112 off after the predetermined period of time. In some embodiments, the predetermined period of time is based upon an amount of time deemed necessary to satisfactorily sanitize a toothbrush and/or a razor with the light source 112. In other embodiments, the circuitry includes a means of allowing a user to adjust the predetermined period of time, the means being a mechanical timer, a digital timer, and/or any other means of entering and/or adjusting a pre-determined period of time.

In other embodiments, the base member includes a back panel 150 that defines one or more slot 152. Each slot 152 is configured to receive one or more suction cup. In this way, the base 100 is capable of being removably mounted to a wall, such as in a shower. In such embodiments, the base is hermetically sealed to protect the power source 110, the light source 112, and/or the circuitry from moisture.

The present invention also includes a method of sanitizing and/or otherwise preserving a first object, such as a toothbrush and/or a razor. The method includes pouring a first fluid through a top opening 218 of a bottom portion 210 of a first container 202 into a first interior area 250 of the bottom portion 210 of the first container 202 and placing at least part of the first object into the first interior area 250. In some embodiments, the method further includes coupling a second portion 220 of the first container 202 onto the bottom portion 210 of the first container 202 so as to reduce evaporation of the first liquid and/or to otherwise prevent the first fluid from pouring out of the first container 202.

In some embodiments, the present invention further includes a method of sanitizing and/or otherwise preserving second object, such as a toothbrush and/or a razor. The method includes pouring a second fluid through a top opening 218 of a bottom portion 210 of a second container 204 into a first interior area 250 of the bottom portion 210 of the second container 204 and placing at least part of the first object into the first interior area 250. In some embodiments, the method further includes coupling a second portion 220 of the second container 204 onto the bottom portion 210 of the second container 204 so as to reduce evaporation of the second liquid and/or to otherwise prevent the second fluid from pouring out of the second container 204.

In some embodiments, the first and/or second fluid is mouthwash and the first and/or second object is a toothbrush. In some such embodiments, the bristle end of the toothbrush is placed into the first interior area 250 of the first 202 and/or second 204 container such that the bristles of the toothbrush are completely submerged in the mouthwash. In other embodiments, the first and/or second fluid is rubbing alcohol and the first and/or second object is a razor. In some such embodiments, the blade end of the razor is placed into the first interior area 250 of the first 202 and/or second 204 container such that the blades of the razor are completely submerged in the rubbing alcohol.

In some embodiments, the method of sanitizing and/or otherwise preserving a first and/or second object further includes shining a sanitizing light 112 towards the first and/or second object. In some such embodiments, the sanitizing light 112 is electrically coupled to circuitry that is capable of shutting off the sanitizing light 112 after a predetermined period of time. In other embodiments, the method of sanitizing and/or otherwise preserving a first and/or second object further includes establishing a period of time for which a sanitizing light 112 is to be directed towards the first and/or second object.

In the foregoing description, certain terms have been used for brevity, clearness and understanding; but no unnecessary limitations are to be implied therefrom beyond the requirements of the prior art, because such terms are used for descriptive purposes and are intended to be broadly construed. Moreover, the description and illustration of the inventions is by way of example, and the scope of the inventions is not limited to the exact details shown or described.

Although the foregoing detailed description of the present invention has been described by reference to an exemplary embodiment, and the best mode contemplated for carrying out the present invention has been shown and described, it will be understood that certain changes, modification or variations may be made in embodying the above invention, and in the construction thereof, other than those specifically set forth herein, may be achieved by those skilled in the art without departing from the spirit and scope of the invention, and that such changes, modification or variations are to be considered as being within the overall scope of the present invention. Therefore, it is contemplated to cover the present invention and any and all changes, modifications, variations, or equivalents that fall with in the true spirit and scope of the underlying principles disclosed and claimed herein. Consequently, the scope of the present invention is intended to be limited only by the attached claims, all matter contained in the above description and shown in the accompanying drawings shall be interpreted as illustrative and not in a limiting sense.

Having now described the features, discoveries and principles of the invention, the manner in which the invention is constructed and used, the characteristics of the construction, and advantageous, new and useful results obtained; the new and useful structures, devices, elements, arrangements, parts and combinations, are set forth in the appended claims.

It is also to be understood that the following claims are intended to cover all of the generic and specific features of the invention herein described, and all statements of the scope of the invention which, as a matter of language, might be said to fall therebetween.

What is claimed is:

1. A sanitizing system comprising
a base member;
a light source coupled to said base member; and
a first container extending vertically from said base member, said first container having a bottom portion and a lid portion removably coupled to said bottom portion,
wherein said bottom portion of said first container comprises a bottom panel and a continuous sidewall extending upward from said bottom panel to a top lip of said bottom portion so as to define a first interior area, said first interior area being capable of retaining liquid,
wherein said top lip of said bottom portion of said first container defines a top opening in communication with said interior area so as to enable liquid to be poured into and out of said first interior area,
wherein said lid portion of said first container defines a bottom lip, said bottom lip of said lid portion of said first container being configured to selectively couple to said top lip of said bottom portion of said first container so as to minimize evaporation of the liquid,
wherein said light source is a sanitizing light source,
wherein at least some sanitizing light from said light source is directed towards said first container, and
wherein at least a portion of said first container is made from a transparent or semi-transparent material so that at least some of the sanitizing light being directed towards said first container is capable of propagating into said interior area of said first container.

2. The sanitizing system of claim 1, wherein said first container is configured to hold an object with a first portion of the object being positioned entirely within said first interior area of said first container such that the first portion of the object is capable of being completely submerged in liquid within said first interior area of said first container, wherein:
the object is one of a toothbrush or a razor;
the first portion of the object is bristles or blades, respectively; and
the liquid is mouthwash or rubbing alcohol, respectively.

3. The sanitizing system of claim 1, wherein said light source is positioned within said base member directly below said first container.

4. The sanitizing system of claim 3, further comprising a lens coupled to said base member and positioned between said light source and said first container such that light from said light source that propagates into said interior area of said first container first propagates through said lens.

5. The sanitizing system of claim 3, wherein said base member comprises a button in electrical communication with said light source so as to enable the button to turn said light source on and off.

6. The sanitizing system of claim 3, wherein said base member further comprises circuitry in electrical communication with said light source, said circuitry being configured to automatically turn the light source off after a predetermined period of time.

7. The sanitizing system of claim 6, wherein said base member further comprises a means of allowing a user to adjust said predetermined period of time.

8. The sanitizing system of claim 1, wherein said base member includes a recessed area having a bottom surface and sidewalls extending upward from said bottom surface, said recessed area being configured to receive at least a portion of said first container so as to support said first container.

9. The sanitizing system of claim 1, further comprising a second container extending vertically from said base member, said second container having a bottom portion and a lid portion removably coupled to said bottom portion,
wherein said bottom portion of said second container comprises a bottom panel and a continuous sidewall extending upward from said bottom panel to a top lip of said bottom portion so as to define an interior area, said interior area being capable of retaining liquid,
wherein said top lip of said bottom portion of said second container defines a top opening in communication with said interior area so as to enable liquid to be poured into and out of said interior area,
wherein said lid portion of said second container defines a bottom lip, said bottom lip of said lid portion of said second container being configured to selectively couple to said top lip of said bottom portion of said second container so as to minimize evaporation of the liquid,
wherein said light source is a sanitizing light source,
wherein at least some sanitizing light from said light source is directed towards said second container, and
wherein at least a portion of said second container is made from a transparent or semi-transparent material so that at least some of the sanitizing light being directed towards said second container is capable of propagating into said interior area of said second container.

10. The sanitizing system of claim 9, wherein the first container is taller than the second container.

11. The sanitizing system of claim 9, wherein:
said first container is configured to hold a toothbrush with its bristles completely contained within said interior area of said first container such that the bristles of the toothbrush may be completely submerged in liquid, and
said second container is configured to hold a razor with its razor blades completely contained within said interior area of said second container such that the razor blades of the razor may be completely submerged in liquid.

12. The sanitizing system of claim 11, wherein the first container is taller than said second container.

13. The sanitizing system of claim 9, wherein said light source is positioned within said base member directly below said first and second containers.

14. The sanitizing system of claim 9, wherein said base member includes a recessed area having a bottom surface and sidewalls extending upward from said bottom surface, said recessed area being configured to receive at least a portion of said first and second containers so as to support said first and second containers.

15. A method of using the sanitizing system of claim 1 to sanitize an object, the method comprising:
pouring a liquid into the first interior area of the first container;
placing at least part of the object into the interior area of the first container such that at least part of the object is submerged in the liquid;
coupling the upper portion of the first container to the lower portion of the first container;
emitting sanitizing light from the light source such that at least some of the sanitizing light reaches at least part of the submerged portion of the object.

16. The method of claim 15, wherein:
the object is a toothbrush;
the liquid is mouthwash; and
the bristles of the toothbrush are completely submerged in the mouthwash.

17. The method of claim 15, wherein:
the object is a razor;
the liquid is rubbing alcohol; and
the bristles of the toothbrush are completely submerged in the rubbing alcohol.

18. A method of using the sanitizing system of claim 9 to sanitize at least two objects simultaneously, the method comprising:
pouring a first liquid into the interior area of the first container;
pouring a second liquid into the interior area of the second container;
placing at least part of a first object into the interior area of the first container such that at least part of the first object is submerged in the first liquid;
placing at least part of a second object into the interior area of the second container such that at least part of the second object is submerged in the second liquid;
coupling the upper portions of the first and second containers to respective lower portions of the first and second containers;
emitting sanitizing light from the light source such that at least some of the sanitizing light reaches at least part of the submerged portion of the first and second objects.

19. The method of claim 18, wherein:
the first object is a toothbrush;
the first liquid is mouthwash; and
the bristles of the toothbrush are completely submerged in the mouthwash.

20. The method of claim 18, wherein:
the second object is a razor;
the second liquid is rubbing alcohol; and
the blades of the razor are completely submerged in the rubbing alcohol.
